(19) United States
(12) Patent Application Publication
(10) Pub. No.: US 2020/0107755 A1
(43) Pub. Date: Apr. 9, 2020

(54) MEASURING DEVICE AND TRANSMISSION METHOD

(71) Applicants: OMRON HEALTHCARE CO., LTD., Muko-shi (JP); OMRON Corporation, Kyoto-shi (JP)

(72) Inventors: Nobuo KUBO, Kyoto (JP); Toru DENO, Kyoto (JP); Hideki KONDO, Kyoto (JP)

(21) Appl. No.: 16/704,003

(22) Filed: Dec. 5, 2019

Related U.S. Application Data
(63) Continuation of application No. PCT/JP2018/028814, filed on Aug. 1, 2018.

(30) Foreign Application Priority Data
Aug. 9, 2017 (JP) 2017-154754

(51) Int. Cl. A61B 5/11; A61B 5/00; H04W 52/02; H04Q 9/00; H04W 84/18

(52) U.S. Cl. CPC A61B 5/1118; A61B 5/0024; H04W 52/0216; A61B 2560/0209; H04W 84/18; H04Q 2209/826; H04Q 9/00

(57) ABSTRACT

A measuring device according to one aspect of the present invention includes a measurement control unit configured to acquire a measurement result obtained by measuring a quantity related to information on a user using a sensor, a packet generation unit configured to generate a one-way communication packet including the acquired measurement result, an action determination unit configured to determine whether or not the user is taking a specific action, a transmission interval adjusting unit configured to adjust a transmission interval based on a result of a determination by the action determination unit, and a packet transmission unit configured to transmit the packet at the adjusted transmission interval.

### CROSS-REFERENCE TO RELATED APPLICATIONS

This application is a Continuation Application of PCT Application No. PCT/JP2018/028814, filed Aug. 1, 2018 and based upon and claiming the benefit of priority from Japanese Patent Application No. 2017-154754, filed Aug. 9, 2017, the entire contents of all of which are incorporated herein by reference.

### FIELD

The present invention relates to a technique in which a quantity related to given information such as biological information is measured using a sensor and a measurement result thus obtained is transmitted to an external device by one-way communication.

### BACKGROUND

Blood pressure monitors having a function of transferring blood pressure data to a user's mobile terminal have been put on the market. Those blood pressure monitors enable a user to browse his or her blood pressure measurement result on his or her mobile terminal. Typically, a short-range communication technique such as Bluetooth (registered trademark) is used to transmit blood pressure data. Bluetooth communication generally consumes less power than WLAN (Wireless Local Area Network) communication. Bluetooth version 4.0 is also called Bluetooth Low Energy (BLE) and achieves a further reduction of consumed power as compared to previous versions.

BLE supports two-way communication called connection. However, the connection has a problem that operations imposed on a user for pairing are complicated. Furthermore, the connection involves complicated communication procedures, thereby leading to such problems that compatibility problems often occur between a blood pressure monitor and a mobile terminal, each of the blood pressure monitor and the mobile terminal requires high-performance hardware (processor or memory), development/evaluation costs are high, communications start slowly, and so on.

On the other hand, BLE also supports one-way communication called advertising. Japanese Patent No. 5,852,620 discloses a technique in which optional data is included and transmitted in a vacant area of a data field of an advertisement packet for detecting a wireless communication device as a connection partner.

When blood pressure data is transmitted in one-way communication, pairing and subsequent complicated communication procedures are no longer required. Thus, the problems described above can be solved or reduced. The transmission of measurement data such as blood pressure data by one-way communication demands a further reduction of consumed power.

### SUMMARY

A measuring device according to one aspect of the present invention includes, a measurement control unit configured to acquire a measurement result obtained by measuring a quantity related to information on a user using a sensor, a packet generation unit configured to generate a one-way communication packet including the acquired measurement result, an action determination unit configured to determine whether or not the user is taking a specific action, a transmission interval adjusting unit configured to adjust a transmission interval based on a result of a determination by the action determination unit, and a packet transmission unit configured to transmit the packet at the adjusted transmission interval.

According to the above configuration, the packet including the measurement result is transmitted to an external device (for example, a user's mobile terminal) by one-way communication, and a transmission interval of the packet is adjusted depending on a result of the determination as to whether or not a user is taking a specific action. In general, when a user is browsing measurement results on a mobile terminal, it is desired that the mobile terminal stores a measurement result immediately after the measuring device obtains it so that the user can browse the measurement result on the mobile terminal immediately after the measurement. On the other hand, when the user is not browsing measurement results on the mobile terminal, the mobile terminal is not required to store a measurement result immediately after the measuring device obtains it. Therefore, setting a long transmission interval causes no problem when there is no (or a small) possibility that the user browses measurement results on the mobile terminal. For example, a user does not use a mobile terminal during sleep, thereby being free from the possibility that he or she browses measurement results on the mobile terminal. As described above, the determination as to whether or not a user is taking a specific action (for example, sleeping) enables the estimation as to whether or not there is any possibility that the user browses measurement results on the mobile terminal. Accordingly, adjustment of a transmission interval in accordance with a result of the determination as to whether or not a user is taking a specific action enables a transmission interval to be extended when there is no possibility that the user browses measurement results on a mobile terminal. This results in reduced power consumption for transmission.

In the measuring device according to the above aspect, the specific action may be ambulation. The transmission interval adjusting unit adjusts the transmission interval to a first value when the action determination unit determines that the user is ambulating, and adjusts the transmission interval to a second value smaller than the first value when the action determination unit determines that the user is not ambulating.

According to the above configuration, a transmission interval is adjusted in a manner such that a transmission interval is set to long when it is determined that a user is ambulating, and is set to short when it is determined that the user is not ambulating. In general, a user does not use a mobile terminal during ambulation, thereby being free from the possibility that he or she browses measurement results on the mobile terminal. Accordingly, adjustment of a transmission interval in accordance with a result of the determination as to whether or not a user is ambulating enables a transmission interval to be extended when there is no possibility that the user browses measurement results on a mobile terminal. This results in reduced power consumption for transmission.

In the measuring device according to the above aspect, the transmission interval adjusting unit may have access to information on a first time frame, the first time frame being a time frame in which the user is scheduled to be sleeping, and the transmission interval adjustment unit may adjust, when the action determination unit determines that the user is not ambulating, the transmission interval in the first time frame to a third value greater than the second value, and adjust the transmission interval in a second time frame to the second value, the second time frame being different from the first time frame.

According to the above configuration, a transmission interval is adjusted to be long during both a period in which a user is ambulating and a period in which the user is scheduled to be sleeping. This increases a total of periods in which a long transmission interval is set, and further reduces power consumption.

In the measuring device according to the above aspect, the specific action may be the ambulation and sleeping. The transmission interval adjusting unit adjusts the transmission interval to a first value when the action determination unit determines that the user is ambulating, adjusts the transmission interval to a second value smaller than the first value when the action determination unit determines that the user is not ambulating and when the action determination unit determines the user is not sleeping, and adjusts the transmission interval to a third value greater than the second value when the action determination unit determines that the user is sleeping.

According to the above configuration, a transmission interval is set to long during a period in which a user is ambulating and a period in which the user is sleeping. This increases the total of periods in which a long transmission interval is set, and further reduces power consumption.

The measuring device according to the above aspect, the specific action may be operation on the measuring device. The transmission interval adjusting unit adjusts the transmission interval to a first value when the action determination unit determines that the user is not operating the measuring device, and adjusts the transmission interval to a second value smaller than the first value when the action determination unit determines that the user is operating the measuring device.

According to the above configuration, a transmission interval is adjusted in a manner such that a transmission interval is set to long when it is determined that a user is not operating the measuring device, and is set to short when it is determined that the user is operating the measuring device. Often times, a user operates the mobile terminal with no break after operating the measuring device. Accordingly, adjustment of a transmission interval in accordance with a result of the determination as to whether or not a user is operating the measuring device enables a transmission interval to be shortened when there is a strong possibility that the user browses measurement results on the mobile terminal, and to be extended for the rest of the time. As a result, power consumption can be reduced.

The present invention can provide a measuring device and a transmission method by which power consumption can be reduced when a measurement result obtained by measuring information related to a user is transmitted by one-way communication.

### BRIEF DESCRIPTION OF THE DRAWINGS

FIG. 1 is a block diagram illustrating a configuration example of an information management system according to an embodiment of the present invention.

FIG. 2 is a block diagram illustrating an example of a hardware configuration of a measuring device illustrated in FIG. 1.

FIG. 3 is a block diagram illustrating an example of a hardware configuration of an information management device illustrated in FIG. 1.

FIG. 4 is a block diagram illustrating an example of a software configuration of the measuring device illustrated in FIG. 1.

FIG. 5 is a diagram for explaining advertising performed in BLE.

FIG. 6 is a diagram illustrating a data structure of a packet to be transmitted and received in BLE.

FIG. 7 is a diagram illustrating a data structure of a PDU field of an advertisement packet.

FIG. 8 is a block diagram illustrating an example of a software configuration of the information management device illustrated in FIG. 1.

FIG. 9 is a flowchart illustrating an example of a transmission interval adjusting method according to the present embodiment.

FIG. 10 is a flowchart illustrating an example of a transmission method in a normal transmission mode according to the present embodiment.

FIG. 11 is a flowchart illustrating an example of a transmission method in a latest measurement result transmission mode according to the present embodiment.

FIG. 12 is a flowchart illustrating an example of a transmission method in a designated measurement result transmission mode according to the present embodiment.

FIG. 13 is a flowchart illustrating an example of a transmission mode switching method according to the present embodiment.

FIG. 14 is a flowchart illustrating an example of an information management method according to the present embodiment.

FIG. 15 is a block diagram illustrating a configuration of an action determination unit according to a modification of the present embodiment.

### DETAILED DESCRIPTION

Hereinafter, embodiments of the present invention will be described with reference to the drawings.

According to one embodiment of the present embodiment, there are provided a measuring device and a transmission method by which power consumption can be reduced when a measurement result obtained by measuring information related to a user is transmitted by one-way communication.

The present invention adopts the following configuration in order to achieve the above object.

§ 1 Application Example

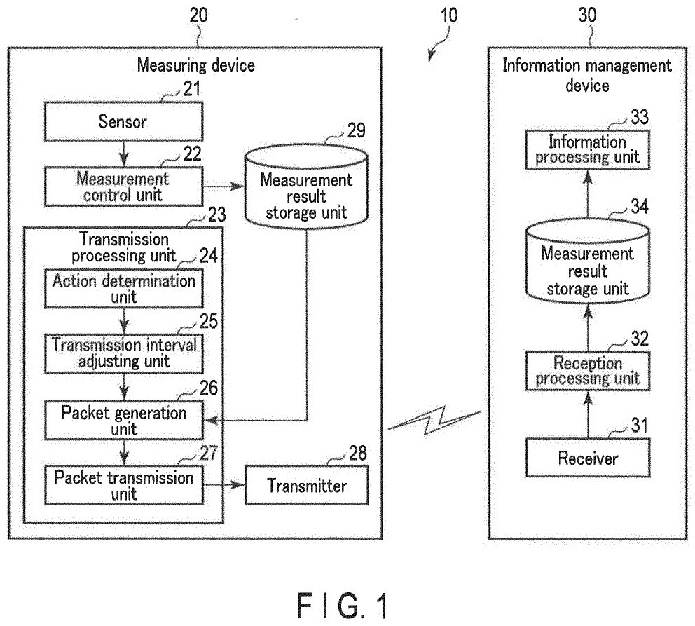

An application example of the present invention will be described with reference to FIG. 1. FIG. 1 illustrates an information management system 10 according to an embodiment. As shown in FIG. 1, the information management system 10 includes a measuring device 20 and an information management device 30. In this application example, the measuring device 20 is, for example, a wearable device worn by a user, while the information management device 30 is, for example, a mobile terminal owned by the user. The mobile terminal may be, for example, a smartphone, a mobile phone, a tablet PC (Personal Computer), a notebook PC, etc.

The measuring device 20 includes a sensor 21 and measures a quantity related to information on the user (hereinafter referred to as user information) using the sensor 21. User information includes, for example, at least one of user's biological information and user's activity information. Biological information means information obtained from the user's body. Examples of biological information include blood pressure, a pulse rate, a heart rate, an electrocardiogram, a body temperature, an arterial oxygen saturation, a blood alcohol concentration, etc. Activity information means information indicating a user's physical activity. Examples of activity information include the number of steps, the number of stair-climbing steps, calorie consumption, etc. Those indicators are also called the amount of activity.

Various types of the sensor 21 are used depending on the type of information on a user to be measured. When a blood pressure value is measured, a pressure sensor, a photoelectric sensor, an ultrasonic sensor, an electrode, or the like is used as the sensor 21. When the number of steps is measured, an acceleration sensor or the like is used as the sensor 21. To simplify explanations, described in the present embodiment is a case in which the measuring device 20 measures a quantity related to one type of user information (for example, blood pressure). However, it should be noted that the measuring device 20 may measure a quantity related to multiple types of user information (for example, a combination of the blood, pressure and the number of steps).

The measuring device 20 further includes a measurement control unit 22, a transmission processing unit 23, a transmitter 28, and a measurement result storage unit 29. The measurement control unit 22 measures a quantity related to user information using the sensor 21, and generates a measurement result indicating the measured quantity related to user information. The measurement control unit 22 stores the generated measurement result in the measurement result storage unit 29. A measurement result is typically associated with measurement time information indicating a measurement time. A measurement result may be further associated with a measurement ID. The measurement ID is a serial number indicating a measurement order. Hereinafter, the measurement ID is also simply referred to as ID.

The transmission processing unit 23 performs processing for transmitting a measurement result and includes an action determination unit 24, a transmission interval adjusting unit 25, a packet generation unit 26, and a packet transmission unit 27.

The action determination unit 24 determines whether or not a user is taking a specific action, and provides a determination result to the transmission interval adjusting unit 25. As one example, the action determination unit 24 determines whether or not the user is ambulating. The action determination unit 24 determines whether or not the user is ambulating, based on an acceleration signal output from the acceleration sensor, for example. Ambulating means a state in which the user is moving on foot. Ambulating includes not only walking but also running.

The transmission interval adjusting unit 25 adjusts a transmission interval for the transmitter 28 based on a determination result by the action determination unit 24. A transmission interval means a time interval between one operation of packet transmission and another operation of packet transmission. For example, the transmission interval adjusting unit 25 adjusts a transmission interval to a first value when the action determination unit 24 determines that a user is ambulating. The transmission interval adjusting unit 25 adjusts a transmission interval to a second value smaller (shorter) than the first value when the action determination unit 24 determines that the user is not ambulating. That is, the transmission interval adjusting unit 25 controls a transmission interval in a manner such that packets are transmitted at low density (i.e., sparsely) when the user is ambulating, and packets are transmitted at high density (that is, densely) when the user is not ambulating.

The packet generation unit 26 reads from the measurement result storage unit 29 a measurement result to transmit, and generates a one-way communication packet containing this particular measurement result. The packet transmission unit 27 transmits packets generated by the packet generation unit 26 at a transmission interval adjusted by the transmission interval adjusting unit 25. Specifically, the packet transmission unit 27 supplies packets to the transmitter 28, and the transmitter 28 wirelessly transmits these packets at a transmission interval adjusted by the transmission interval adjusting unit 25. The transmitter 28 is a transmitter that is sometimes called a beacon terminal, and is configured to periodically transmit wireless signals to surrounding areas. The transmitter 28 may comply with a short-range wireless communication standard such as Bluetooth or Bluetooth Low Energy (BLE).

When the measuring device 20 transmits packets at high density, the information management device 30 can easily receive the packets from the measuring device 20. This enables, when the measuring device 20 obtains a new measurement result, the information management device 30 to receive this measurement result immediately after the measurement.

The information management device 30 manages measurement results obtained by the measuring device 20, and includes a receiver 31, a reception processing unit 32, an information processing unit 33, and a measurement result storage unit 34.

The information management device 30 typically includes a transceiver in compliance with a wireless communication standard that is identical to or compatible with that of the transmitter 28 of the measuring device 20, and the receiver 31 is a part of this transceiver. The receiver 31 receives a packet from the measuring device 20 and provides the received packet to the reception processing unit 32. The reception processing unit 32 extracts a measurement result from the packet and stores it in the measurement result storage unit 34. Since the measuring device 20 transmits the same measurement result many times, the reception processing unit 32 may acquire the same measurement result as that already acquired. In this case, the reception processing unit 32 discards a duplicated measurement result thus obtained, without storing it in the measurement result storage unit 34. The information processing unit 33 processes measurement results stored in the measurement result storage unit 34. For example, the information processing unit 33 presents measurement results to a user by performing statistical processing or graphing.

Generally, when a user is not walking (for example, when the user is sitting), he or she may operate the information management device 30 to browse measurement results thereon. In addition, when the measuring device 20 obtains a new measurement result, the user may desire to check this measurement result obtained by the measuring device 20, on the information management device 30 immediately after the measurement. For this reason, when there is a possibility that a user browses measurement results on the information management device 30, it is desirable that the measuring device 20 transmits packets at high density so that the user can check the latest measurement result immediately on the information management device 30.

On the other hand, a user generally does not operate the information management device 30 when he or she is ambulating. Therefore, there is no (or a small) possibility that a user browses measurement results when he or she is ambulating. Unless there is a possibility that a user browses measurement results, the immediacy described above is not required. Thus, there is no problem with the measuring device 20 transmitting packets at low density.

In the present embodiment, the measuring device 20 adjusts a transmission interval depending on a determination result as to whether or not a user is taking a specific action. In this manner, packets can be transmitted at low density when there is no (or a small) possibility that the user browses measurement results. This results in reduced power consumption for transmission.

Hereinafter, the measuring device 20 and the information management device 30 will be described in more detail. In the example described below, the measuring device 20 is a blood pressure monitor of a wristwatch type, and measures a blood pressure on a wrist as a site to be measured. Note that a site to be measured is not limited to the wrist, but may be another site such as the upper arm.

§ 2 Structural Example

(Hardware Structure)

<Measuring Device>

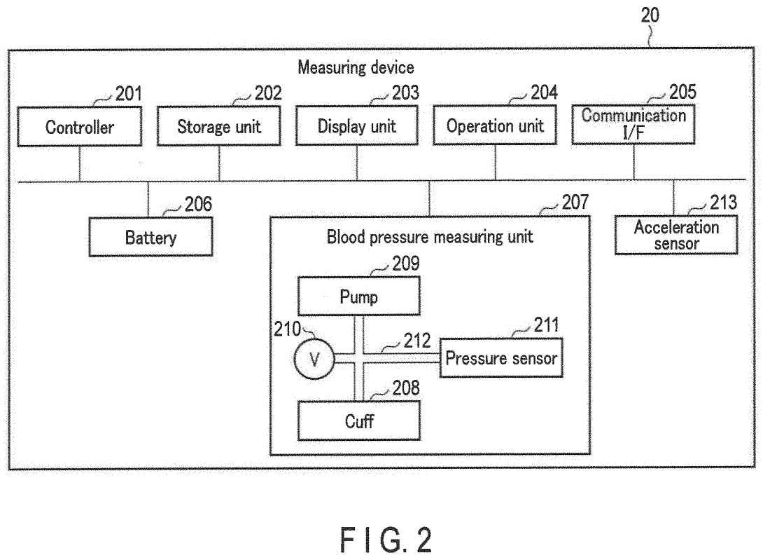

FIG. 2 illustrates an example of a hardware configuration of the measuring device 20. As illustrated in FIG. 2, the measuring device 20 includes a controller 201, a storage unit 202, a display unit 203, an operation unit 204, a communication interface 205, a battery 206, a blood pressure measuring unit 207, and an acceleration sensor 213.

The controller 201 includes a central processing unit (CPU), a random access memory (RAM), a read only memory (ROM), etc., and controls each structural element according to information processing. The storage unit 202 is an auxiliary storage device such as a semiconductor memory (for example, a flash memory). The storage unit 202 stores a blood pressure measurement program to be executed by the controller 201, measurement result data indicating a blood pressure value calculated by the controller 201, etc. The blood pressure measurement program is a program for causing the measuring device 20 to measure a user's blood pressure.

The display unit 203 displays information such as a measurement result. As the display unit 203, for example, a liquid crystal display (LCD), an organic light emitting diode (OLED) display, etc., can be used. The operation unit 204 allows a user to input an instruction directed to the measuring device 20. The operation unit 204 provides the controller 201 with an instruction signal in accordance with an operation by a user. The operation unit 204 includes, for example, a plurality of push buttons. As a combination of the display unit 203 and the operation unit 204, a touch screen may be used.

In the present embodiment, the operation unit 204 includes the first to third buttons. The first button is used to switch screens. The second button is used to indicate a determination. The third button is used to indicate cursor movement. For example, when the first button is pressed by a user while a home screen is displayed on the display unit 203, the display unit 203 is caused to display a screen for confirming whether or not to perform blood pressure measurement. When the second button is pressed while the confirmation screen is displayed, the measuring device 20 performs blood pressure measurement. Furthermore, when the first button is pressed while the confirmation screen is displayed, the display unit 203 is caused to display a screen for browsing a measurement result history. The history browsing screen includes, for example, a list of measurement results (for example, a list of measurement IDs or measurement times). A user moves a cursor to a desired measurement result using the third button, and presses the second button. This causes the display unit 203 to display details of this particular measurement result. When the first button is pressed while the history browsing screen is displayed, the display unit 203 is caused to display the home screen.

The communication interface 205 is an interface for communicating with an external device. In this embodiment, the communication interface 205 includes only a transmitter that broadcasts radio signals at predetermined transmission intervals. That is, the communication interface 205 has a transmission function but no reception function. The transmitter performs transmission processing including up-conversion and amplification. A transmitter that consumes low power is desirable. In the present embodiment, the communication interface 205 complies with BLE, and uses a communication method called advertising that broadcasts signals without connecting to any network. An interval between the transmissions described above corresponds to a so-called advertising interval in BLE. An advertising interval indicates a time interval between one advertising communication and another advertising communication. The advertising interval can be set in units of 0.625 [ms] in the range of 20 [ms] to 10.24 [s]. For advertising communication, three channels called advertising channels are used. In one advertising communication, signals are transmitted using the three channels sequentially.

In another embodiment, the communication interface 205 may further include a communication module that enables bidirectional communication. The communication module may be a wireless communication module or a wired communication module, or may include both of them.

The battery 206 is a rechargeable secondary battery, for example. The battery 206 supplies power to each structural element in the measuring device 20. The battery 206 supplies power to the controller 201, the storage unit 202, the display unit 203, the operation unit 204, the communication interface 205, and the blood pressure measuring unit 207, for example.

The blood pressure measuring unit; 207 measures a user's blood pressure. In the example shown in FIG. 2, the blood pressure measuring unit 207 includes a cuff 208, a pump 209, an exhaust valve 210, and a pressure sensor 211. The cuff 208 includes an air bag. The air bag is connected to the pump 209 and the exhaust valve 210 via an air passage 212. The pump 209 supplies air to the air bag of the cuff 208. When air is supplied to the air bag by the pump 209, the air bag is inflated. The inflation of the air bag causes the cuff 208 to press a site to be measured (in this example, a wrist). The exhaust valve 210 is provided to exhaust air from the air hag of the cuff 208. Driving of the pump 209 and both opening and closing of the exhaust valve 210 are controlled by the controller 201. The pressure sensor 211 detects pressure inside the cuff 208 and outputs a pressure signal indicating the detected pressure to the controller 201. The controller 201 calculates a blood pressure value based on the pressure signal received from the pressure sensor 211. Blood pressure values include, but are not limited to, systolic blood pressure (SBP) and diastolic blood pressure (DBP).

Although not shown in FIG. 2, an amplifier configured to amplify an output signal of the pressure sensor 211, and an analog-to-digital converter configured to convert an output signal of the amplifier from an analog signal to a digital signal, are provided between the pressure sensor 211 and the controller 201.

The acceleration sensor 213 is, for example, a three-axis acceleration sensor, and outputs an acceleration signal indicative of acceleration in the three mutually orthogonal directions.

Regarding a specific hardware configuration of the measuring device 20, structural elements can be omitted, replaced, and added as appropriate according to the embodiment. For example, the controller 201 may include a plurality of processors.

<Information Management Device>

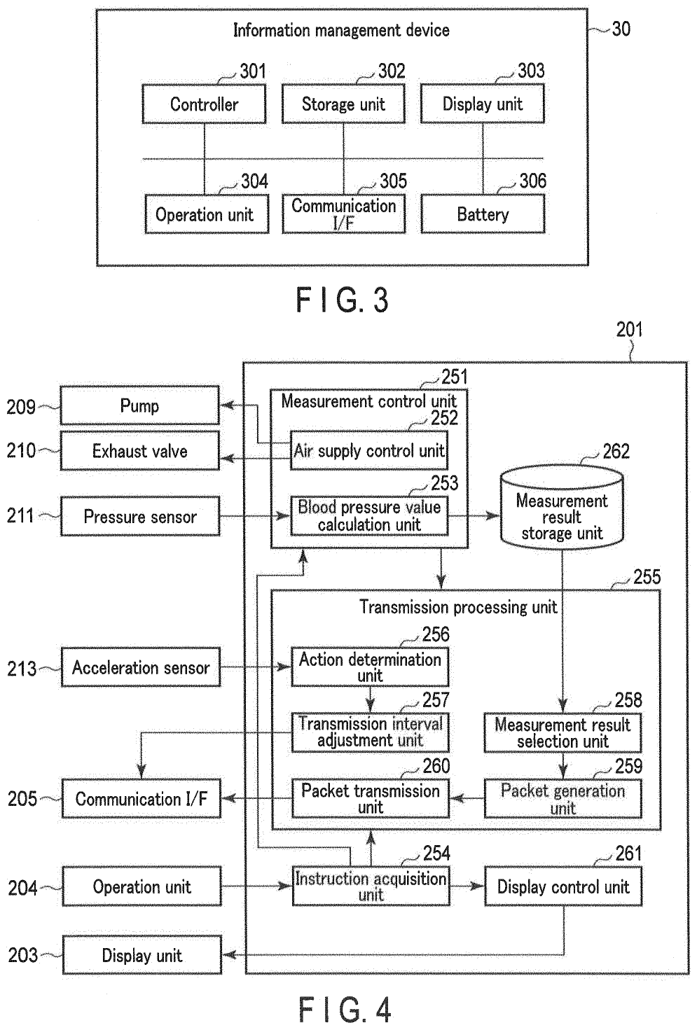

FIG. 3 illustrates an example of a hardware configuration of the information management device 30. As illustrated in FIG. 3, the information management device 30 includes a controller 301, a storage unit 302, a display unit 303, an operation unit 304, a communication interface 305, and a battery 306.

The controller 301 includes a CPU, a RAM, a. ROM, etc., and controls each structural element in accordance with information processing. The storage unit 302 is an auxiliary storage device such as a hard disk drive (HDD), a semiconductor memory (for example, a solid state drive (SSD)), etc. The storage unit 302 stores an information management program to be executed by the controller 301, data on measurement results received from the measuring device 20, etc. The information management program is a program for causing the measuring device 20 to manage measurement results.

The combination of the display unit 303 and the operation unit 304 is realized by a touch screen. The touch screen may be of a pressure-sensitive (resistive) type or a proximity (capacitance) type. As the display unit 303, for example, an LCD, an OLED display, etc., can be used. The operation unit 304 enables a user to input an instruction directed to the information management device 30. The operation unit 304 provides the controller 301 with an instruction signal in accordance with an operation by the user. The operation unit 304 may further include a plurality of push buttons. The display unit 303 and the operation unit 304 may be realized as separate devices.

The communication interface 305 is an interface for communicating with an external device. The communication interface 305 includes a wireless communication module corresponding to a wireless communication standard that is identical to or compatible with that of the communication interface 205 of the measuring device 20. This wireless communication module performs reception processing including amplification and down-conversion on a received signal. In the present embodiment, the communication interface 305 includes a BLE communication module. This BLE communication module is also usable for bidirectional communication with an external device independent of the measuring device 20. The communication interface 305 may further include an additional wireless communication module. For example, the communication interface 305 includes a Wi-Fi (registered trademark) module, is connected to a network (for example, the Internet) via a Wi-Fi base station, and communicates with an external device via the network. The communication interface 305 may further include a wired communication module. For example, the communication interface 305 may include a USE connector and connect to an external device via a USE cable.

The battery 305 is a rechargeable secondary battery, for example. The battery 306 supplies power to each structural element within the information management device 30. The battery 306 supplies power to the controller 301, the storage unit 302, the display unit 303, the operation unit 304, and the communication interface 305, for example.

Regarding a specific hardware configuration of the information management device 30, structural elements can be omitted, replaced, and added as appropriate according to the embodiment. For example, the controller 301 may include a plurality of processors. Furthermore, the information management device 30 may be realized by a plurality of information processing devices (computers).

(Software Configuration)

<Measuring Device>

With reference to FIG. 4, an example of a software configuration of the measuring device 20 will be described.

The controller 201 of the measuring device 20 (FIG. 2) loads into the RAM, a blood pressure measurement program stored in the storage unit 202. Then, the controller 201 causes the CPU to interpret and execute the blood pressure measurement program loaded into the RAM, thereby controlling each structural element. In this manner, as illustrated in FIG. 4, the measuring device 20 functions as a computer including a measurement control unit 251, an instruction acquisition unit 254, a transmission processing unit 255, a display control unit 261, and a measurement result storage unit 262. The measurement result storage unit 262 is realized by the storage unit 202.

The measurement control unit 251 measures a user's blood pressure. In one example, the measurement control unit 251 starts measurement when a recommended condition for measuring blood pressure is satisfied. Such conditions include, for example, a condition that a present time reaches a preset time (for example, 7:30 a.m. and 10:30 p.m.). In another example, the measurement control unit 251 starts measurement in response to a user's operation.

The measurement control unit 251 includes an air supply control unit 252 and a blood pressure value calculation unit 253. The air supply control unit 252 controls the supply of fluid to the cuff 208. Specifically, the air supply control unit 252 controls driving of the pump 209 and both opening and closing of the exhaust valve 210. The blood pressure value calculation unit 253 calculates a blood pressure value by an oscillometric method based on a pressure signal received from the pressure sensor 211, in a pressurizing process for supplying air to the cuff 208 or a depressurizing process for exhausting air from the cuff 208. A pulse rate can be calculated simultaneously with a blood pressure value. The blood pressure value calculation unit 253 stores a measurement result indicating the calculated blood pressure value in the measurement result storage unit 262 in a manner such that the measurement result is associated with measurement time information and the measurement ID.

The instruction acquisition unit 254 acquires an instruction input by a user using the operation unit 204. Examples of the instruction include an instruction to start measurement, an instruction for browsing measurement result history, etc. Upon acquisition of an instruction to start measurement, the instruction acquisition unit 254 provides this instruction to the measurement control unit 251. Upon acquisition of a history browsing instruction, the instruction acquisition unit 254 provides this instruction to the display control unit 261.

The display control unit 261 controls the operation of the display unit 203. The display control unit 261 changes the display content in response to a user's operation. In addition, immediately after a new measurement result is obtained, the display control unit 261 causes the display unit 203 to display this new measurement result.

The transmission processing unit 255 reads from the measurement result storage unit 262 a plurality of measurement results to transmit, and generates a plurality of packets each containing a corresponding one of the read measurement results. The transmission processing unit 255 transmits the generated packets via the communication interface 205. The transmission processing unit 255 may read from the measurement result storage unit 262 one measurement result to transmit, and generates and transmits a packet containing this particular measurement result.

The transmission processing unit 255 includes a measurement result selection unit 256, an action determination unit 257, a transmission interval adjusting unit 258, a packet generation unit 259, and a packet transmission unit 260. The transmission processing unit 255 has a plurality of transmission modes. In the present embodiment, the transmission processing unit 255 has three transmission modes, i.e., a normal transmission mode, a latest measurement result transmission mode, and a designated measurement result transmission mode. The transmission processing unit 255 may have only one transmission mode (for example, the normal transmission mode).

First, a case in which the transmission processing unit 255 operates in the normal transmission mode will be described.

The action determination unit 257 determines whether or not a user is taking a specific action, and provides a determination result to the transmission interval adjusting unit 258. The action determination unit 257 determines whether or not the user is taking a specific action, based on an acceleration signal output from the acceleration sensor 213, for example. As one example, the action determination unit 257 determines, based on an acceleration signal, whether or not the user is ambulating. In another example, the action determination unit 257 determines, based on an acceleration signal, whether or not the user is sleeping. Specifically, the action determination unit 257 detects, based on an acceleration signal, a movement such as rolling over, and based on a result of this detection, determines whether or not the user is sleeping. The action determination unit 257 may use other types of sensors (such as a microphone) instead of or in addition to the acceleration sensor 213.

The transmission interval adjusting unit 258 adjusts a transmission interval based on a result of a determination by the action determination unit 257. As one example, the transmission interval adjusting unit 258 adjusts a transmission interval to the first value (for example, one second) when the action determination unit 257 determines that a user is ambulating. The transmission interval adjusting unit 258 adjusts a transmission interval to the second value (for example, 160 milliseconds) smaller than the first value when the action determination unit 257 determines that the user is not ambulating. In another example, the transmission interval adjusting unit 258 adjusts a transmission interval to a first value when the action determination unit 257 determines that a user is sleeping. The transmission interval adjusting unit 258 adjusts a transmission interval to a second value smaller than the first value when the action determination unit 257 determines that the user is not sleeping. The first value and the second value are variable. For example, the first value and the second value may be changed in accordance with a user's operation. Furthermore, the first value and the second value may be changed in accordance with remaining battery charge. The first value and the second value may be fixed values.

The measurement result selection unit 256 selects a plurality of measurement results to transmit, from measurement results stored in the measurement result storage unit 262. In one example, the measurement result selection unit 256 selects a predetermined number of measurement results in order from the latest measurement result. In another example, the measurement result selection unit 256 selects measurement results obtained during a predetermined time period (for example, measurement results acquired during the most recent week). Selection processing is not limited to these examples. The measurement result selection unit 256 may perform selection processing every time a new measurement result is obtained, or periodically.

The packet generation unit 259 generates one or more packets based on the plurality of measurement results selected by the measurement result selection unit 256, and provides the plurality of generated packets to the packet transmission unit 260. A plurality of measurement results selected by the measurement result selection unit 256 are assigned to packets, respectively. The packet transmission unit 260 transmits, via the communication interface 205, packets generated by the packet generation unit 259 at transmission intervals determined by the transmission interval adjusting unit 258.

Transmission processing of the transmission processing unit 255 will be described with a specific example. Herein, it is assumed that three measurement results, measurement result 1, measurement result 2, and measurement result 3, are transmitted. The packet generation unit 259 generates three packets, i.e., packet 1 including measurement result 1, packet 2 including measurement result 2, and packet 3 including measurement result 3. The packet transmission unit 260 repeats the operation of transmitting packet 1, packet 2, and packet 3 in this order. That is, the packet transmission unit 260 sequentially transmits packet 1, packet 2, and packet 3 in a manner to transmit packet 1, packet 2, packet 3, packet 1, packet 2, packet 3, packet 1, . . . . In this way, the measuring device 20 can repeatedly transmit the plurality of measurement results.

Each packet may include a plurality of measurement results. In the case of including two measurement results in each packet, for example, the packet generation unit 26 may generate two packets, i.e., packet 1 including measurement result 1 and measurement result 2, and packet 2 including measurement result 1 and measurement result 3. In another example, the transmission processing unit 23 may generate three packets, i.e., packet 1 including measurement result 1 and measurement result 2, packet 2 including measurement result 1 and measurement result 3, and packet 3 including measurement result 2 and measurement result 3. The packet generation unit 26 may generate one packet including measurement result 1, measurement result 2, and measurement result 3.

The information management system 10 may have a situation in which the information management device 30 fails to receive a packet from the measuring device 20. This situation occurs because, for example, the information management device 30 is away from the measuring device 20, the information management device 30 is turned off, the wireless communication function of the information management device 30 is turned off, and so on. Assume that the measuring device 20 transmits only a measurement result obtained by the first measurement during a period between the first measurement and the second measurement (in this case, only a measurement result obtained by the second measurement is transmitted during a period between the second measurement and the next third measurement). In this case, unless the information management device 30 receives the measurement result obtained by the first measurement from the measuring device 20 during this particular period, the information management device 30 loses an opportunity to receive this measurement result. In some cases, some degree of data loss may be allowed to occur on the information management device 30; however, in many cases, it is preferable that the information management device 30 receives all measurement results obtained by the measuring device 20.

In the present embodiment, the measuring device 20 transmits a plurality of measurement results in one-way communication packets in a manner such that measurement results including the first measurement result and measurement results obtained before the first measurement result are transmitted during the first period from when the first measurement result is obtained until the next second measurement result is obtained, and that measurement results including the first measurement result and the second measurement result transmitted during the second period from when the second measurement result is obtained until the next third measurement result is obtained. That is, the first measurement result is transmitted during not only the first period but also the second period. Accordingly, even if the first measurement result is not received by the information management device 30 during the first period, it can still be received by the information management device 30 during the second period. In the above case, each of measurement results is transmitted over a longer period of time and thus has a higher probability of being successfully received by the information management device 30 as compared to a case in which only the latest measurement result is transmitted (in this case, only the first measurement result is transmitted during the first period, and only the second measurement is transmitted during the second period). As a result, the occurrence of data loss can be reduced in the information management device 30.

Next, a case in which the transmission processing unit 255 operates in the latest measurement result transmission mode will be described.

The latest measurement result transmission mode is a mode in which the latest measurement result generated by the measurement control unit 251 is intensively transmitted. At the end of blood pressure measurement, a measurement result obtained by this measurement has not been transmitted yet, and thus the information management device 30 has not received this measurement result. The transmission processing unit 255 operates in the latest measurement result transmission mode and intensively transmits the latest measurement result until a predetermined time elapses after the latest measurement result is generated by the measurement control unit 251. This facilitates the information management device 30's reception of the latest measurement result, and allows a user to browse the latest measurement result with the information management device 30 immediately after the measurement.

When the blood pressure measurement is completed, the measurement control unit 251 transmits to the transmission processing unit 255, measurement completion information indicating that the latest measurement result has been obtained. When the transmission processing unit 255 receives the measurement completion information from the measurement control unit 251, the transmission mode is switched from the normal transmission mode to the latest measurement result transmission mode. In the present embodiment, the transmission processing unit 255 operating in the latest measurement result transmission mode transmits only the latest measurement result. In another embodiment, the transmission processing unit 255 operating in the latest measurement result transmission mode may transmit a plurality of measurement results including the latest measurement result.

The action determination unit 257 and the transmission interval adjusting unit 258 perform operations similar to those described in connection with the normal transmission mode. For example, the action determination unit 257 estimates whether or not a user is ambulating. The transmission interval adjusting unit 256 adjusts a transmission interval to a first value when the action determination unit 257 determines that the user is ambulating. The transmission interval adjusting unit 258 adjusts a transmission interval to a second value smaller than the first value when the action determination unit 257 determines that the user is not ambulating. Regardless of the determination result by the action determination unit 257, the transmission interval adjusting unit 258 may adjust a transmission interval to a value smaller than the first value. This value may be equal to or different from the second value.

The measurement result selection unit 255 selects the latest measurement result from measurement results stored in the measurement result storage unit 262. The packet generation unit 259 generates a packet including the latest measurement result selected by the measurement result selection unit 256. The packet transmission unit 260 transmits this packet at a transmission interval adjusted by the transmission interval adjusting unit 258. When a certain period of time elapses after the completion of blood pressure measurement (or after a transmission mode is switched to the latest measurement result transmission mode), the transmission mode returns to the normal transmission mode.

Next, a case in which the transmission processing unit 255 operates in the designated measurement result transmission mode will be described.

The designated measurement result transmission mode is a mode in which a measurement result designated by a user is intensively transmitted. When a user is browsing a measurement result history on the measuring device 20, the transmission processing unit 255 operates in the designated measurement result transmission mode. A user inputs an instruction using the operation unit 204, and a measurement result designated by this instruction is displayed on the display unit 203. During a period in which the designated measurement result is displayed on the display unit 203, the transmission processing unit 255 operates in the designated measurement result transmission mode, and intensively transmits the designated measurement result. This makes it easy for the information management device 30 to receive the measurement result designated by a user. For example, measurement information that has not been received by the information management device 30 is specified. As a result, the data loss can be solved in the information management device 30. A user's operation for causing the display unit 203 to display thereon a specific measurement result corresponds to an instruction for causing the measuring device 20 to transmit this specific measurement result. In the present embodiment, the transmission processing unit 255 operating in the designated measurement result transmission mode transmits only a measurement result designated by a user (that is, a measurement result displayed on the display unit 203). In another embodiment, the transmission processing unit 255 operating in the designated measurement result transmission mode may transmit a plurality of measurement results including a measurement result designated by the user.

The action determination unit 257 and the transmission interval adjusting unit 258 perform operations similar to those described in connection with the normal transmission mode. For example, the action determination unit 257 estimates whether or not a user is ambulating. The transmission interval adjusting unit 258 adjusts a transmission interval to a first value when the action determination unit 257 determines that a user is ambulating. The transmission interval adjusting unit 258 adjusts a transmission interval to a second value smaller than the first value when the action determination unit 257 determines that a user is not ambulating. Regardless of a result of the determination by the action determination unit 257, the transmission interval adjusting unit 258 may adjust a transmission interval to a value smaller than the first value. This value may be equal to or different from the second value

Hereinafter, advertisement of BLE will be schematically described.

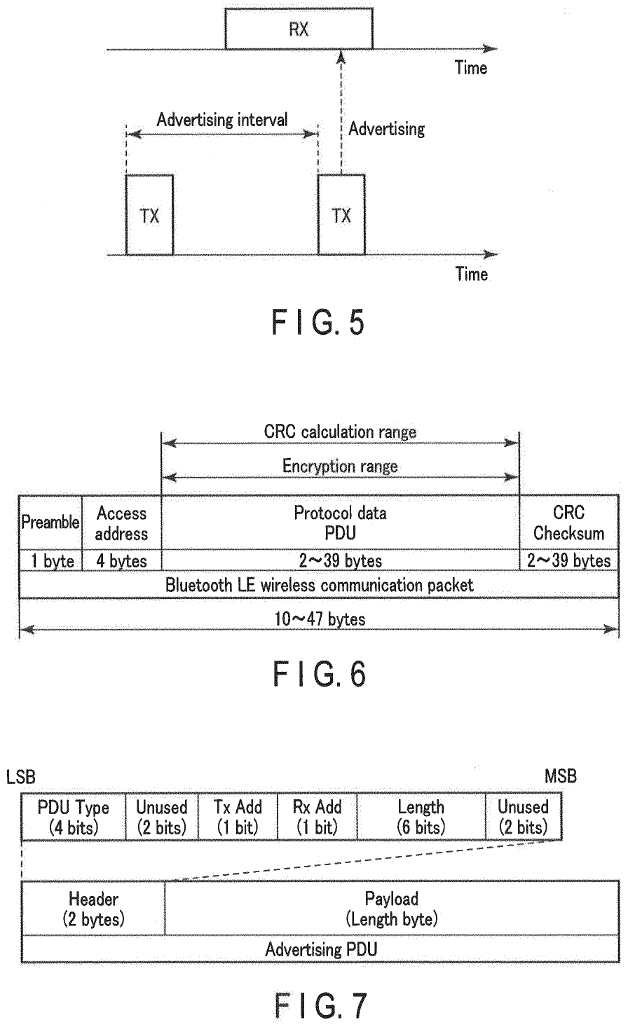

In a passive scan method employed in BLE, as illustrated in FIG. 5, a new node periodically transmits advertisement packets for informing its own presence. This new node can save power consumption by entering a sleep state between one transmission and a next transmission of advertisement packets. In addition, since an advertisement packet receiving side also operates intermittently, the transmission and reception of advertisement packets involve very small power consumption.

FIG. 6 illustrates a basic configuration of a BLE wireless communication packet. A BLE wireless communication packet includes a 1-byte preamble, a 4-byte access address, a 2 to 39-byte (variable) protocol data unit (PDU), and a 3-byte cyclic redundancy checksum (CRC). The length of BLE wireless communication packet depends on the length of the PDU, and is 10 to 47 bytes.

The preamble field is prepared for synchronization of BLE wireless communication and stores the repetition of “01” or “10”. The access address stores a fixed numerical value for an advertising channel and a random access address for a data channel. The present embodiment targets an advertisement packet that is a BLE wireless communication packet transmitted on the advertising channel. The CRC field is used for the detection of reception errors. The CRC calculation range includes only the PDU field.

Next, the PDU field of the advertisement packet will be described with reference to FIG. 7. The PDU field of a data communication packet, which is a BLE wireless communication packet transmitted on a data channel, has a data structure different from that shown in FIG. 7. However, the description of such a PDU packet is omitted because the present embodiment does not target a data communication packet.

The PDU field of an advertisement packet includes a 2-byte header and a 0 to 37-byte (variable) payload. The header further includes a 4-bit PDU Type field, a 2-bit unused field, a 1-bit TxAdd field, a 1-bit RxAdd field, a 6-bit Length field, and a 2-bit unused field.

The PDU Typefield stores a value indicating a type of this particular PDU. Some values such as “connectable advertising” and “non-connecting advertising” are predefined. The TxAdd field stores a flag indicating whether or not a transmission address is present in the payload. Similarly, the RxAdd field stores a flag indicating whether or not a reception address is present in the payload. The Length field stores a value indicating a byte size of the payload. Given data can be stored in the payload. Accordingly, the measuring device 20 stores a measurement result (SBP and DBP in this example), measurement time information, and a measurement ID in the payload using a predetermined data structure. The payload may further include an identifier representing the measuring device 20 as a transmission source device.

Described in the present embodiment is an example in which all the functions of the measuring device 20 are realized by a general-purpose CPU. However, part or all of the above functions may be realized by one or more dedicated processors.

<Information Management Device>

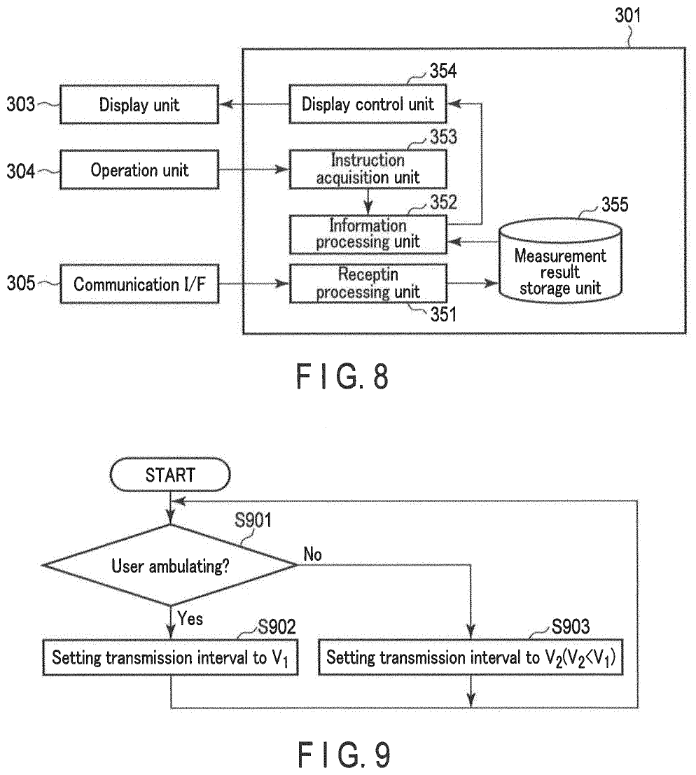

With reference to FIG. 8, an example of a software configuration of the information management device 30 according to the present embodiment will be described.

The controller 301 of the information management device 30 (FIG. 3) loads into the RAM, a lifestyle management program stored in the storage unit 302. Then, the controller 301 causes the CPU to interpret and execute the lifestyle management program loaded into the RAM, thereby controlling each structural element. In this manner, as illustrated in FIG. 8, the information management device 30 functions as a computer including a reception processing unit 351, an information processing unit 352, an instruction acquisition unit 353, a display control unit 354, and a measurement result storage unit 355. The measurement result storage unit 355 is realized by the storage unit 302.

The reception processing unit 351 receives a packet from the measuring device 20 via the communication interface 305. The reception processing unit 351 confirms an identifier included in the packet, and discards the received packet when a value of the identifier is inappropriate. When a value of the identifier is appropriate, the reception processing unit 351 extracts a measurement result, measurement time information, and a measurement ID included in the packet, and stores them in the measurement result storage unit 355.

The information processing unit 352 processes measurement results stored in the measurement result storage unit 355. For example, the information processing unit 352 graphs measurement results. Furthermore, the information processing unit 352 determines the presence or absence of data loss, that is, whether or not there is any unreceived measurement result. The information processing unit 352 determines the presence or absence of data loss by, for example, confirming the continuity of measurement IDs. A specific example of the determination method will be described later. The information management device 30 is incapable of informing the measuring device 20 of data loss even if it is detected, because the communication between the measuring device 20 and the information management device 30 is one-way communication from the measuring device 20 to the information management device 30. For this reason, the information management device 30 presents (for example, displays) the presence of unreceived measurement result to a user. Information to be presented includes a measurement ID of an unreceived measurement result. In this manner, the user is encouraged to input to the measuring device 20 an instruction for transmitting an unreceived measurement result.

The information processing unit 352 may not have a function of determining the presence or absence of data loss. In such a case, a user may discover data loss when he or she is browsing measurement results on the information management device 30.

The instruction acquisition unit 353 acquires an instruction input by a user using the operation unit 204 and passes this instruction to the information processing unit 352. Examples of the instruction include an instruction for displaying a measurement result, etc. The display control unit 354 controls the operation of the display unit 303. For example, the display control unit 354 generates image data including a graph generated by the information processing unit 352 and provides the image data to the display unit 303.

Described in the present embodiment is an example in which all the functions of the information management device 30 are realized by a general-purpose CPU. However, part or all of the above functions may be realized by one or more dedicated processors.

§ 3 Example of Operation

<Measuring Device>

An operation example of the measuring device 20 according to the present embodiment will be described.

FIG. 9 illustrates an example of a transmission interval adjusting operation performed by the measuring device 20. The processing shown in FIG. 9 starts when the measuring device 20 is turned on. In step S901, the controller 201 of the measuring device 20 functions as the action determination unit 257 and determines whether or not a user is ambulating.

When the controller 201 determines in step S901 that the user is ambulating, the processing proceeds to step S902. In step S902, the controller 201 functions as the transmission interval adjusting unit 258 and sets a transmission interval to first value V₁. Thereafter, the processing returns to step S901.

When the controller 201 determines in step S901 that the user is not ambulating, the processing proceeds to step S903. In step S903, the controller 201 functions as the transmission interval adjusting unit 258 and sets a transmission interval to second value V₂ (V₂<V₁). Thereafter, the processing returns to step S901.

In this way, the controller 201 controls a transmission interval in a manner such that a transmission interval is set to the first value V₁ while the user is ambulating, and is set to the second value V₂ smaller than the first value V₁ while the user is not ambulating.

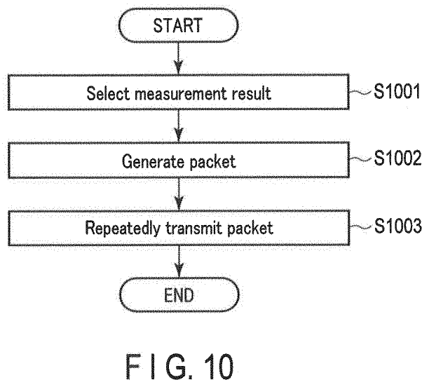

FIG. 10 Illustrates an example of a transmission operation of the measuring device 20 in the normal transmission mode. The transmission operation illustrated in FIG. 10 starts, for example, when the transmission mode is switched to the normal transmission mode. In step S1001 of FIG. 10, the controller 201 functions as the measurement result selection unit 256 and selects a plurality of measurement results to transmit, from measurement results stored in the storage unit 202 (specifically, the measurement result storage unit 262). For example, the controller 201 selects two measurement results, that is, measurement result 1 and measurement result 2 (the latest measurement result at the present time) obtained after measurement result 1.

In step S1002, the controller 201 functions as the packet generation unit 259, and generates a plurality of packets based on the selected measurement results. Each packet includes at least one of the selected measurement results. For example, the controller 201 generates packet 1 including measurement result 1 and packet 2 including measurement result 2. In step S1003, the controller 201 functions as the packet transmission unit 260, and transmits the generated packets at transmission intervals adjusted in accordance with the processing shown in FIG. 9. The processing shown in step S1003 is continued until, for example, the transmission mode is switched. For example, the controller 201 repeats the operation of transmitting packet 1 and packet 2. When measurement result 3 subsequent to measurement result 2 is obtained, the transmission mode is switched to the latest measurement result transmission mode and is then restored to the normal transmission mode, as will be described later. At this time, the controller 201 repeats the operation of transmitting packet 2 including measurement result 2 and packet 3 including measurement result 3.

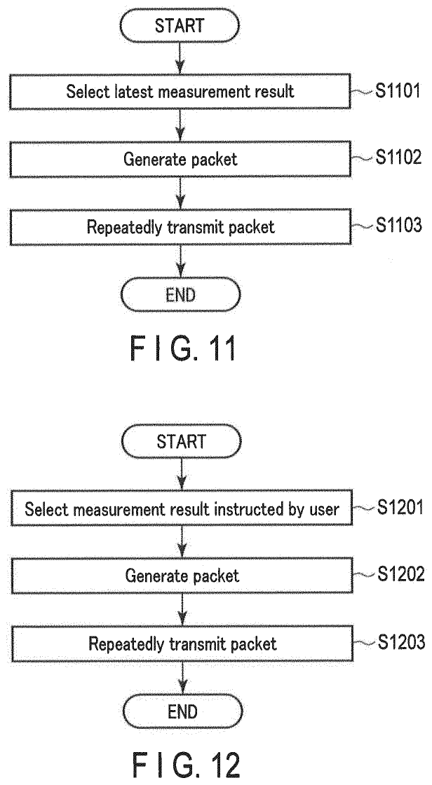

FIG. 11 illustrates an example of a transmission operation of the measuring device 20 in the latest measurement result transmission mode. The transmission operation illustrated in FIG. 11 starts when the transmission mode is switched to the latest measurement result transmission mode. In step S1101 of FIG. 11, the controller 201 functions as the measurement result selection unit 256 and selects the latest measurement result from the measurement results stored in the storage unit 202. In step S1102, the controller 201 functions as the packet generation unit 259 and generates a packet including the latest selected measurement result. In step S3110, the controller 201 functions as the packet transmission unit 260, and transmits the generated packets at transmission intervals adjusted in accordance with the processing shown in FIG. 9. The processing shown in step S1103 is continued until, for example, the transmission mode is switched.

FIG. 12 illustrates an example of a transmission operation of the measuring device 20 in a designated measurement result transmission mode. The transmission operation illustrated in FIG. 12 starts when the transmission mode is switched to the designated measurement result transmission mode. In step S1201 of FIG. 12, the controller 201 functions as the measurement result selection unit 256 and selects a measurement result designated by a user, from measurement results stored in the storage unit 202. In step S1202, the controller 201 functions as the packet generation unit 259 and generates a packet including the selected measurement result. In step S1203, the controller 201 functions as the packet transmission unit 260, and transmits the generated packets at transmission intervals adjusted in accordance with the processing shown in FIG. 9. The processing shown in step S1203 is continued until, for example, the transmission, mode is switched.

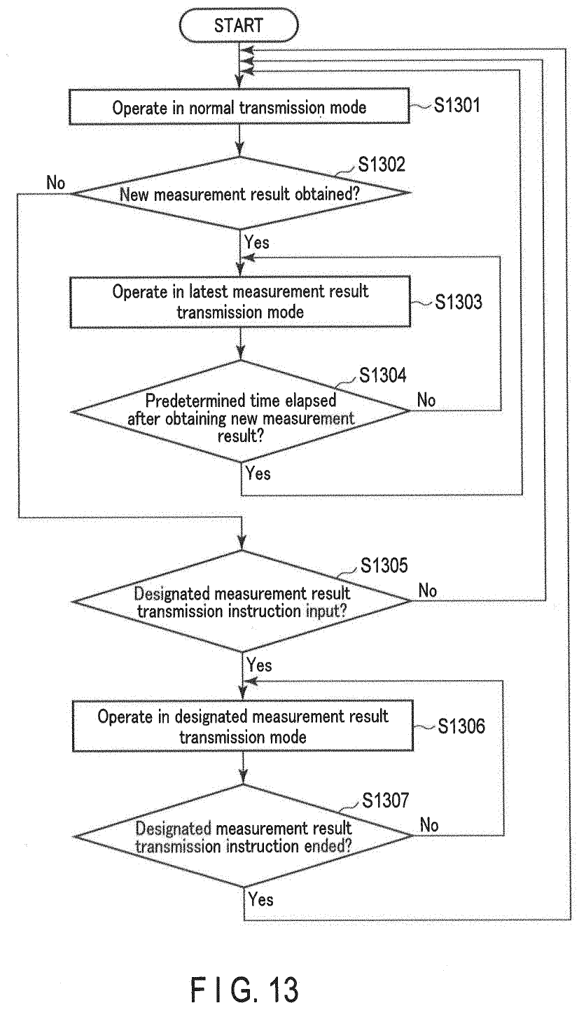

FIG. 13 illustrates an example of the transmission mode switching operation of the measuring device 20. When the measuring device 20 is powered on, first, the transmission mode is set to the normal transmission mode. In step S1301 of FIG. 13, the controller 201 operates in the normal transmission mode. In the normal transmission mode, the controller 201 performs the processing described above with reference to FIG. 10.

In step S1302, the controller 201 determines whether or not a new measurement result is obtained. If no new measurement result is obtained, the processing proceeds to step S1305. If a new measurement result is obtained, the processing proceeds to step S1303.

In step S1303, the transmission mode is switched from the normal transmission mode to the latest measurement result transmission mode. In the latest measurement result transmission mode, the controller 201 performs the processing described above with reference to FIG. 11. In step S1304, the controller 201 determines whether or not a predetermined time (for example, 5 minutes) has elapsed after the obtainment of a new measurement result. Until a predetermined time has elapsed after the obtainment of a new measurement result, the controller 201 operates in the latest measurement result transmission mode. When a predetermined time elapses after the obtainment of a new measurement result, the processing returns to step S1301, and the transmission mode is switched from the latest measurement result transmission mode to the normal transmission mode.

If the processing proceeds from step S1302 to step S1305, the controller 201 determines in step S1305 whether or not a user has input an instruction for transmitting a specific measurement result (designated measurement result transmission instruction). If no designated measurement result transmission instruction is input by a user, the processing returns to step S1301. If a designated measurement result transmission instruction is input by a user, the processing proceeds to step S1306. The designated measurement result transmission instruction corresponds to the fact that a user operates the operation unit 204 so as to cause the display unit 203 to display a specific measurement result.

In step S1306, the transmission mode is switched from the normal transmission mode to the designated measurement result transmission mode. In the designated measurement result transmission mode, the controller 201 performs the processing described above with reference to FIG. 12,

In step S1307, the controller 201 determines whether or not the designated measurement result transmission instruction has ended. For example, when a user inputs an instruction to switch from a screen for confirming a history of measurement results to a home screen, the controller 201 determines that the designated measurement result transmission instruction has ended. Until the designated measurement result transmission instruction ends, the controller 201 operates in the designated measurement result transmission mode. If the designated measurement result transmission instruction ends, the processing returns to step S1301 and the transmission mode is switched from the designated measurement result transmission mode to the normal transmission mode.

The processing procedure described above is merely an example, and each processing may be changed to the extent possible. Furthermore, in the processing procedure described above, steps can be omitted, replaced, and added as appropriate according to the embodiment. For example, in the transmission mode switching operation shown in FIG. 13, even the controller 201 operating in the latest measurement result mode may determine whether or not a user has input a designated measurement result transmission instruction. If a user inputs the designated measurement result transmission instruction while the controller 201 is operating in the latest measurement result mode, the transmission mode is switched from the latest measurement result transmission mode to the designated measurement result transmission mode.

Described in the present embodiment is an example in which all the functions of the measuring device 20 are realized by a general-purpose CPU. However, part or all of the above functions may be realized by one or more dedicated processors.

<Information Management Device>

An operation example of the information management device 30 according to the present embodiment will be described.

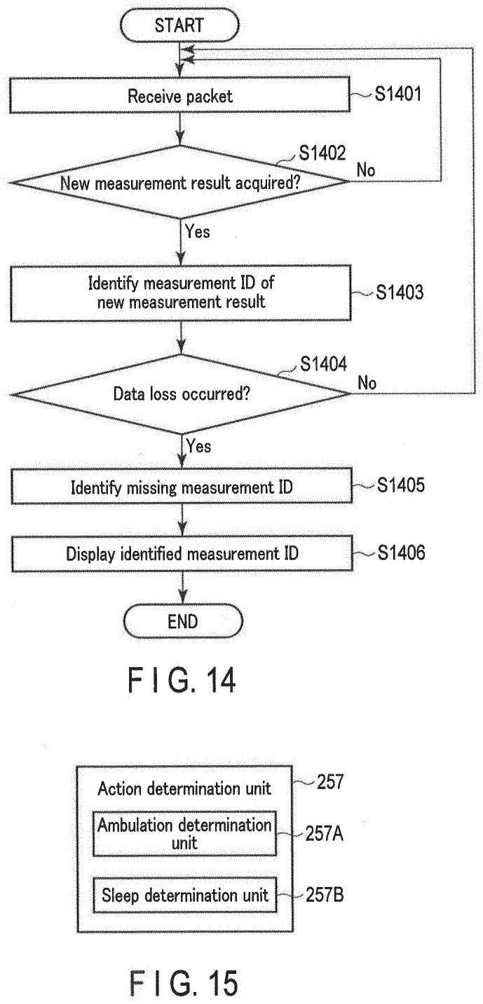

FIG. 14 illustrates an example of a processing procedure of the information management device 30. This example assumes that the measuring device 20 is designed to transmit 10 measurement results in order from the latest.

In step S1401 shown in FIG. 14, the controller 301 of the information management device 30 functions as a reception processing unit 351, receives a packet from the measuring device 20 via the communication interface 305, and obtains a measurement result included in the received packet. In step S1402, the controller 301 functions as the information processing unit 352, and determines whether or not the obtained measurement result is a new measurement result (a measurement result that has not been received so far). If the obtained measurement result is not new, the processing returns to step S1401, and the controller 301 receives a next packet. If a new measurement result is received from the measuring device 20, the processing proceeds to step S1403. In step S1403, the controller 301 functions as the information processing unit 352 and identifies an ID of the received new measurement result.

In step S1404, the controller 301 functions as the information processing unit 352, and makes a determination with respect to a set of IDs that are smaller than the identified ID by 10 or more, whether or not data loss has occurred. If the measuring device 20 transmits 10 newer measurement results, any measurement result assigned with an ID that is smaller than the identified ID by 10 or more is not transmitted in the normal transmission mode. In other words, the information management device 30 misses the chance to receive a measurement result assigned with an ID that is smaller than the identified ID by 10 or more. For this reason, in order to solve the data loss, a user needs to instruct the measuring device 20 to transmit a measurement result that has not been received by the information management device 30. If data loss has occurred, the processing proceeds to step S1405. If no data loss has occurred, the processing returns to step S1401. As an example, in the case of a new measurement result being assigned with ID of 257, the controller 301 determines whether or not all the measurement results assigned with IDs 1 to 247 are present in the measurement result storage unit 355. If all the measurement results assigned with IDs 1 to 247 are present, the controller 301 determines that no data loss has occurred. If not, the controller 301 determines that data loss has occurred.

In step S1405, the controller 301 functions as the information processing unit 352 and identifies a missing ID. In step S1406, the controller 301 functions as the display control unit 354 and causes the display unit 303 to display information indicating the identified ID. In the above example, for example, if the measurement result assigned with ID=247 is absent in the measurement result storage unit 355, the controller 301 causes the display unit 303 to display the message that “measurement result with ID 247 has not been received”. A user confirms the information displayed on the display unit 303 and inputs to the measuring device 20, an instruction for transmitting a measurement result that has not been received by the information management device 30.

After the execution of the processing shown in step S1406, the operational procedure shown in FIG. 14 is started again.

In this way, the information management device 30 presents to a user, information indicating an unreceived measurement result. This encourages a user to input to the measuring device 20, an instruction for causing the measuring device 20 to transmit a measurement result that has not been received by the information management device 30. In response to a user's operation, the measuring device 20 transmits a measurement result that has not been received by the information management device 30, and the information management device 30 receives this measurement result. As a result, data loss can be solved in the information management device 30.

Described in the present embodiment is an example in which all the functions of the information management device 30 are realized by a general-purpose CPU. However, part or all of the above functions may be realized by one or more dedicated processors.

### Effect

In the present embodiment, the measuring device 20 according to the present embodiment adjusts a transmission interval of packets depending on whether or not a user is taking a predetermined action. For example, when a user is ambulating, the measuring device 20 sees no possibility of the user browsing measurement results on the information management device 30 and extends a transmission interval. This reduces power consumption for transmission. When the user is not ambulating, the measuring device 20 sees the possibility of the user browsing measurement results on the information management device 30 and shortens a transmission interval. This enables a measurement result obtained by the measuring device 20 to be checked on the information management device 30 immediately after the measurement. As a result, power consumption can be reduced while meeting a demand to check a measurement result obtained by the measuring device 20, on the information management device 30 immediately after the measurement.

In the present embodiment, measurement results are transmitted by one-way communication. This frees a user from complicated pre-setting such as pairing in Bluetooth. As a result, usability can be improved. Furthermore, this case does not require each of the measuring device 20 and the information management device 30 to execute a complicated communication procedure. Thus, this case has advantages over the case of using two-way communication, in that hardware resources such as a processor and a memory can be saved and development/evaluation costs can be reduced.

§ 4 Modification

The action determination unit 257 may estimate whether or not a user is operating the measuring device 20, based on an acceleration signal from the acceleration sensor 213. When operating the measuring device 20, a user who wears the measuring device 20 on his or her left arm takes a posture with the left arm bent so that the user can view the display unit 203 of the measuring device 20. The action determination unit 257 assumes that the user who is taking such a posture is operating the measuring device 20. The action determination unit 257 may use an angular velocity sensor instead of or in addition to the acceleration sensor 213. The action determination unit 257 may determine that a period from when a user inputs an instruction the operation unit 204 until a certain time elapses is a period in which the user is operating the measuring device 20.

The transmission interval adjusting unit 258 adjusts a transmission interval to a first value when the action determination unit 257 determines that a user is not operating the measuring device 20. The transmission interval adjusting unit 258 adjusts a transmission interval to a second value smaller than the first value when the action determination unit 257 determines that the user is operating the measuring device 20. Often times, a user operates a mobile terminal with no break after operating the measuring device 20. Adjustment of a transmission interval in accordance with a result of the determination as to whether or not a user is operating the measuring device 20 enables a transmission interval to be shortened when there is a certain degree of possibility that the user browses measurement results on the mobile terminal, and to be extended when there is no possibility or a low possibility that the user browses measurement results on the mobile terminal. As a result, power consumption can be reduced.

A transmission interval may be adjusted depending on whether or not a user is taking a predetermined action, and further, depending on the time frame. While a user is sleeping, there is no possibility that the user browses measurement results on the information management device 30. Therefore, the time frame in which the user is scheduled to be sleeping can be deemed to be free from the possibility that he or she browses measurement results on the information management device 30. The transmission interval adjusting unit 258 has access to information on a time frame in which the user is scheduled to be sleeping. The case in which the transmission interval adjusting unit 258 has access to information on a time frame in which the user is scheduled to be sleeping includes the case in which, when the user inputs a scheduled sleeping time frame, the storage unit 202 stores the input time frame so that the transmission interval adjusting unit 258 can access information on the input time frame. The transmission interval adjusting unit 258 adjusts a transmission interval to a first value when the action determination unit 257 determines that the user is ambulating. When the action determination unit 257 determines that the user is not ambulating, the transmission interval adjusting unit 258 adjusts a transmission interval to a third value greater (longer) than a second value during a time frame (for example, from 10:30 p.m. to 7:00 a.m.) in which the user is scheduled to be sleeping, and adjusts a transmission interval to the second value during the rest of the time frames (for example, from 7:00 a.m. to 11:30 p.m.). This increases a total of periods in which a long transmission interval is set. As a result, power consumption can be further reduced. Whether or not a present time is included in a time frame in which the user is scheduled to be sleeping can be determined using a timer incorporated into the information management device 30.

In some examples described above, the action determination unit 257 makes a determination as to one type of action. The action determination unit 257 may make a determination as to a plurality of types of action. FIG. 15 illustrates a configuration of the action determination unit 257 according to a modification.

The action determination unit 257 shown in FIG. 15 includes an ambulation determination unit 257A and a sleep determination unit 257B. The ambulation determination unit 257A determines whether or not the user is ambulating. The sleep determination unit 257B determines whether or not the user is sleeping.

When the ambulation determination unit 257A determines that the user is ambulating, the transmission interval adjusting unit 258 adjusts a transmission interval to the first value. When the ambulation determination unit 257A determines that the user is not ambulating, and the sleep determination unit 257B determines that the user is not sleeping (that is, the user is awake), the transmission interval adjusting unit 258 adjusts a transmission interval to a second value smaller than the first value. When the sleep determination unit 257B determines that the user is sleeping, the transmission interval adjusting unit 258 adjusts a transmission interval to a third value smaller than the second value. The third value may be equal to or different from the first value. This increases a total of periods in which a long transmission interval is set. As a result, power consumption can be further reduced.

In the present embodiment described above, the measuring device 20 measures blood pressure using the oscillometric method. The measuring device 20 may measure the blood pressure by other methods. The measuring device 20 may be a blood pressure measuring apparatus configured to obtain a blood pressure value on a beat by beat basis. For example, the measuring device 20 may measure blood pressure by the tonometry method. The measuring device 20 may detect a pulse transit time (PTT), which is a propagation time of a pulse wave propagating through an artery, using two or more electrodes, and estimate a blood pressure value (for example, SBP and DBP) based on the detected pulse transit time. The measuring device 20 may measure a volume pulse wave optically and estimate a blood pressure value based on the measurement result. Furthermore, the measuring device 20 may measure blood pressure using ultrasonic waves.

In the present embodiment described above, a plurality of measurement results selected by the measurement result selection unit 256 are transmitted at the same transmission ratio. A transmission ratio indicates a transmission ratio of each measurement result to the entire measurement results selected by the measurement result selection unit 256. A transmission ratio is expressed in fractions, decimals, integers, etc. A plurality of measurement results selected by the measurement result selection unit 256 may be transmitted at different transmission ratios. In the selected measurement results, a newer measurement result is less likely to be successfully received by the information management device 30, whereas an older measurement result is more likely to be successfully received by the information management device 30. A higher transmission ratio is set to a newer measurement result. This facilitates the reception of a new measurement result by the information management device 30.

Information stored in a payload of a packet may be encrypted. As an example, the measuring device 20 causes the display unit 203 to display an encryption key used for encryption, and a user confirms the encryption key and inputs it to the information management device 30 using the operation unit 304. The controller 301 of the information management device 30 decrypts the payload portion of the packet using this encryption key. This enables the transmission of a measurement result from the measuring device 20 to the information management device 30 without concern for information leakage. An encryption key may be changed periodically.

A quantity (e.g., a physical quantity) to be measured is not limited to a quantity related to user information. For example, a quantity to be measured may be a quantity related to the environment such as temperature or radiation dose.

In short, the present invention is not limited to the above-described embodiments and can be embodied in practice by modifying the structural elements without departing from the gist of the invention. In addition, various inventions can be made by suitably combining the structural elements disclosed in connection with the above embodiments. For example, some of all the structural elements described in the embodiments may be omitted. In addition, the structural elements between different embodiments may be combined as appropriate.

§ 5 Additional Note

Part or all of the above-mentioned embodiments may also be described as in the following additional notes, without limitation thereto.

(Additional Note 1)

A measuring device comprising:

at least one processor; and

a memory connected to the at least one processor, wherein the at least one processor is configured to:

acquire a measurement result obtained by measuring a quantity related to information on a user using a sensor;

generate a one-way communication packet including the acquired measurement result;

determine whether or not the user is taking a specific action;

adjust a transmission interval based on a determination result; and

transmit the packet at the adjusted transmission interval.

(Additional Note 2)

A transmission method performed by a measuring device configured to obtain a measurement result by measuring a quantity related to information on a user using a sensor, the transmission method comprising:

generating, by at least one processor, a one-way communication packet including the measurement result;

determining, by at least one processor, whether or not the user is taking a specific action;

adjusting, by using at least one processor, a transmission interval based on a determination result; and

transmitting, by at least one processor, the packet at the adjusted transmission interval.

### REFERENCE SIGNS LIST

10 Information management system

20 Measuring device

21 Sensor

22 Measurement control unit

23 Transmission processing unit

24 Action determination unit

25 Transmission interval adjusting unit

26 Packet generation unit

27 Packet transmission unit

28 Transmitter

29 Measurement result storage unit

201Controller

202 Storage unit

203 Display unit

204 Operation unit

205 Communication interface

206 Battery

207 Blood pressure measuring unit

208 Cuff

209 Pump

210 Exhaust valve

211 Pressure sensor

212 Air passage

213 Acceleration sensor

251 Measurement control unit

252 Air supply control unit

253 Blood pressure value calculation unit

254 Instruction acquisition unit

255 Transmission processing unit

256 Measurement result selection unit

257 Action determination unit

257A Ambulation determination unit

257B Sleep determination unit

258 Transmission interval adjusting unit

259 Packet generation unit

260 Packet transmission unit

261 Display control unit

262 Measurement result storage unit

30 Information management device

31 Receiver

32 Reception processing unit

33 Information processing unit

34 Measurement result storage unit

301 Controller

302 Storage unit

303 Display unit

304 Operation unit

305 Communication interface

306 Battery

351 Reception processing unit

352 Information processing section

353 Instruction acquisition unit

354 Display control unit

355 Measurement result storage unit

1. A measuring device comprising:
at least one processor configured to:
acquire a measurement result obtained by measuring a quantity related to biological information on a user using a first sensor;
generate a one-way communication packet including the acquired measurement result;
determine whether the user is ambulating or has stopped ambulating, by measuring a quantity related to activity information on the user using a second sensor;
adjust a transmission interval to a first value when it is determined that the user is ambulating, and adjust the transmission interval to a second value smaller than the first value when it is determined that the user has stopped ambulating; and
transmit the packet at the adjusted transmission interval.

2. The measuring device according to claim 1, wherein the at least one processor is further configured to:
access information on a first time frame, the first time frame being a time frame in which the user is scheduled to be sleeping; and
adjust, when it is determined that the user has stopped ambulating, the transmission interval in the first time frame to a third value greater than the second value, and adjust the transmission interval in a second time frame to the second value, the second time frame being different from the first time frame.

3. The measuring device according to claim 1, wherein the at least one processor is further configured to:
determine whether or not the user is sleeping; and
adjust the transmission interval to the first value when it is determined that the user is ambulating, adjust the transmission interval to a second value smaller than the first value when it is determined that the user has stopped ambulating and the user is not sleeping, and adjust the transmission interval to a third value greater than the second value when it is determined that the user is sleeping.

4. A transmission method performed by a measuring device configured to obtain a measurement result by measuring a quantity related to biological information on a user using a first sensor, the transmission method comprising:
generating a one-way communication packet including the measurement result;
determining whether the user is ambulating or has stopped ambulating, by measuring a quantity related to activity information on the user using a second sensor;
adjusting a transmission interval to a first value when it is determined that the user is ambulating, and adjusting the transmission interval to a second value smaller than the first value when it is determined that the user has stopped ambulating; and
transmitting the packet at the adjusted transmission interval.

5. A non-transitory computer readable medium including computer executable instructions, wherein the instructions, when executed by a processor, cause the processor to perform a method comprising:
generating a one-way communication packet including a measurement result, the measurement result being obtained by measuring a quantity related to biological information on a user using a first sensor;
determining whether the user is ambulating or has stopped ambulating, by measuring a quantity related to activity information on the user using a second sensor;
adjusting a transmission interval to a first value when it is determined that the user is ambulating, and adjusting the transmission interval to a second value smaller than the first value when it is determined that the user has stopped ambulating; and
transmitting the packet at the adjusted transmission interval.
